(19) United States
(12) Patent Application Publication
(10) Pub. No.: US 2022/0409229 A1
(43) Pub. Date: Dec. 29, 2022

(54) MEDICAL TREATMENT TOOL AND ELECTROMAGNETIC WAVE MEDICAL SYSTEM

(71) Applicant: National University Corporation Shiga University of Medical Science, Shiga (JP)

(72) Inventors: Tohru TANI, Shiga (JP); Shigeyuki NAKA, Shiga (JP); Atsushi YAMADA, Shiga (JP)

(73) Assignee: National University Corporation Shiga University of Medical Science, Shiga (JP)

(21) Appl. No.: 17/620,765

(22) PCT Filed: Jun. 20, 2020
(86) PCT No.: PCT/JP2020/024270

§ 371 Date: Dec. 20, 2021

(30) Foreign Application Priority Data
Jun. 21, 2019 (JP) 2019-115046

(51) Int. Cl. A61B 17/3201; A61B 18/18

(52) U.S. Cl. CPC A61B 17/3201; A61B 18/1815; A61B 2018/00083

(57) ABSTRACT

Provided is a scissors-type medical treatment tool capable of not only local tissue fixation in an incision/cutting part but also heating on side surfaces of electrodes of a forceps/tweezers type. It has been found out that a tissue part in which tissue is nipped between blades and a tissue part which is brought into abutment against side surfaces of the blades can be heated simultaneously or sequentially by optimizing arrangement of microwave application electrodes and microwave receiver electrodes in a blade pair (in particular, by causing coagulation to be performed evenly on the left and right of the blades), and the present invention has therefore been achieved.

### TECHNICAL FIELD

The present invention relates to a medical treatment tool and an electromagnetic wave medical system which are capable of electromagnetic wave heating from a side surface of a blade or a forceps jaw.

The present application claims priority from Japanese Patent Application No. 2019-115046, the disclosure of which is incorporated herein by reference.

### BACKGROUND ART

(Device Using Microwave)

There has been known that a microwave can coagulate (immobilize) biological tissue such as digestive organs, a liver, a bladder, a prostate, a uterus, blood vessels, or intestinal tracts at low temperature. Then, various devices for assisting surgery through use of the microwave have been developed.

### RELATED ART

As the devices using a microwave, the following plurality of devices have been reported.

In Patent Literature 1, there is disclosed “a medical treatment tool including: a first electrode including one or a plurality of microwave application antennas 1 and one or a plurality of microwave receiver antennas 1; a second electrode including one or a plurality of microwave application antennas 2 and one or a plurality of microwave receiver antennas 2; a first coaxial cable including a first central conductor and a first external conductor, the first central conductor and the first external conductor being connected directly or indirectly to the microwave application antenna 1 and the microwave receiver antenna 1, respectively; a second coaxial cable including a second central conductor and a second external conductor, the second central conductor and the second external conductor being connected directly or indirectly to the microwave application antenna 2 and the microwave receiver antenna 2, respectively; a microwave transmission coaxial cable; and a microwave splitter for splitting a microwave transmitted by the microwave transmission coaxial cable to the first central conductor and the second central conductor, in which the medical treatment tool can irradiate a microwave from both the first electrode and the second electrode.”

In this Patent Literature, a microwave distribution circuit is disclosed, but a configuration of the medical treatment tool according to the present invention is not disclosed.

In Patent Literature 2, there is disclosed “a power distribution circuit including two distribution lines which are branched into two in the vicinity of an input terminal having electrical continuity to an input line and have electrical continuity to corresponding output lines at respective output terminals, the power distribution circuit including: two connection lines having electrical continuity to the output terminals of the two distribution lines, respectively; an isolation resistor arranged between the two connection lines; and a stub provided at a branch portion of the two distribution lines.”

In this Patent Literature, the configuration of the medical treatment instrument according to the present invention is not disclosed.

In Patent Literature 3, there is disclosed “a microwave surgical instrument including: a surgical instrument main body including an electrode portion for irradiating biological tissue with a microwave; a microwave oscillator which is provided in the surgical instrument main body, and is configured to oscillate a microwave; and an amplifier which is provided in the surgical instrument main body, and is connected between the electrode portion and the microwave oscillator, the amplifier being configured to amplify the microwave from the microwave oscillator and send the amplified microwave to the electrode portion, the microwave surgical instrument further including: a variable output matching circuit which is connected between the amplifier and the electrode portion, and is configured to match an output impedance of the amplifier and an impedance of the biological tissue with each other; a detection circuit configured to separately detect reflected power and incident power given between the amplifier and the electrode portion; and control means for controlling the variable output matching circuit based on values of the incident power and the reflected power detected by the detection circuit.”

In this Patent Literature, capability of electromagnetic wave (in particular, microwave) heating from a side surface of a blade of the medical treatment tool according to the present invention and a configuration of the blade are not disclosed.

In Patent Literature 4, there is disclosed “electrosurgical forceps including: a pair of jaw elements pivotable relative to each other to open and close a gap therebetween; a first transmission line structure mounted in one of the pair of jaw elements adjacent to the gap; a second transmission line structure mounted in the other one of the pair of jaw elements adjacent to the gap opposite the first transmission line structure; a coaxial cable for conveying microwave frequency energy; and a power splitter at a distal end of the coaxial cable, the power splitter being arranged to divide the microwave frequency energy conveyed by the coaxial cable between the first transmission line structure and the second transmission line structure, in which each of the first transmission line structure and the second transmission line structure consists of an unbalanced lossy transmission line to support the microwave energy as a travelling wave, and in which each of the first transmission line structure and the second transmission line structure has an electrical length along the travelling wave that is non-resonant for the microwave energy.”

In this Patent Literature, the configuration of the blade of the medical treatment instrument according to the present invention is not disclosed.

As described above, in the related-art patent literatures, a structure (in particular, structure capable of microwave heating from the side surface of the blade) of the medical treatment tool according to the present invention is not disclosed or suggested.

### CITATION LIST

### Patent Literature

[PTL 1] WO2018/147243A1
[PTL 2] JP 11-330813 A
[PTL 3] JP 2012-115384 A
[PTL 4] JP 2016-533862 A

### SUMMARY OF INVENTION

### Technical Problem

When dielectric heating using a microwave which is a type of electromagnetic wave is used, tissue between electrodes can be subjected to tissue fixation uniformly at low temperature. In addition, the heating can be performed between the electrodes or locally in the vicinity, and hence treatment can be performed solely on a part to be heated or an area of pathology, which is advantageous in that normal tissue is not affected. However, in general, a scissors type is capable of heating only a part in which the tissue is nipped between the electrodes, and a tweezers type is capable of heating only a part in which the electrode is applied to the tissue. Therefore, a hemostatic ability is slightly impaired when, for example, parenchymal organs (e.g., liver, kidney, and spleen) having tissue in contact with a side surface of a blade are subjected to tissue fixation (protein coagulation or hemostasis) and incision. A swift hemostatic process has required switching between the scissors type and the tweezers type, thereby inhibiting swift treatment and continuous treatment. In particular, it has been possible to heat the tissue satisfactorily on the place near the side of a blade including an antenna, but it has not been possible to sufficiently heat the tissue on the side of a blade including no antenna, and there has been a difference in state of heating the tissue between the blade on the antenna side and the blade including no antenna.

Therefore, the present invention has been made in view of the above-mentioned problems, and is aimed to provide a medical treatment tool capable of not only local tissue fixation in an incision/cutting part but also fixation of tissue (including tissue having a forceps tip buried in the tissue) through heating on side surfaces of electrodes of a forceps/tweezers type.

### Solution to Problem

As a result of repeating extensive research to solve the above-mentioned problems, the inventors of the present invention have found out that a tissue part in which tissue is nipped between blades and a tissue part which is brought into abutment against side surfaces of the blades can be heated (dielectrically heated) simultaneously or sequentially by optimizing arrangement of electromagnetic wave application electrodes (in particular, microwave application electrodes) and electromagnetic wave receiver electrodes (in particular, microwave receiver electrodes) in a blade pair, and has achieved the present invention.

Through understanding that living tissue to be treated is a dielectric body and is a member that forms a microwave heating circuit, it has been made possible to optimally arrange the microwave application electrode and the microwave receiver electrode.

More specifically, it has been made possible to widen a coagulation width by utilizing such a property that pinpoint coagulation at a blade edge tip of a related-art blade close to an utmost limit flies uniformly to a certain distance of a microwave.

That is, the present invention includes the following aspects.

1. A medical treatment tool, including:

a first blade or a first jaw including a first electromagnetic wave application electrode and a first electromagnetic wave receiver electrode;

a second blade or a second jaw, which is arranged so as to be opposed to the first blade or the first jaw, and includes a second electromagnetic wave application electrode and a second electromagnetic wave receiver electrode; and

a coaxial cable including a central conductor and an external conductor,

wherein the central conductor is directly or indirectly connected to the first electromagnetic wave application electrode and the second electromagnetic wave application electrode, and the external conductor is directly or indirectly connected to the second electromagnetic wave receiver electrode and the second electromagnetic wave receiver electrode,

wherein the first electromagnetic wave application electrode is located on an upper side of the first blade or the first jaw with respect to the first electromagnetic wave receiver electrode (or the first electromagnetic wave application electrode is located on a side farther from a line (hereinafter referred to sometimes as “tangential line”) in which the first blade or the first jaw and the second blade or the second jaw face each other, with respect to the first electromagnetic wave receiver electrode) and the second electromagnetic wave application electrode is located on an upper side of the second blade or the second jaw with respect to the second electromagnetic wave receiver electrode (or the second electromagnetic wave application electrode is located on a side closer to the line in which the first blade or the first jaw and the second blade or the second jaw face each other, with respect to the second electromagnetic wave receiver electrode), or the first electromagnetic wave receiver electrode is located on the upper side of the first blade or the first jaw with respect to the first electromagnetic wave application electrode (or the first electromagnetic wave application electrode is located on the side closer to the line in which the first blade or the first jaw and the second blade or the second jaw face each other, with respect to the first electromagnetic wave receiver electrode) and the second electromagnetic wave receiver electrode is located on the upper side of the second blade or the second jaw with respect to the second electromagnetic wave application electrode (or the second electromagnetic wave application electrode is located on the side farther from the line in which the first blade or the first jaw and the second blade or the second jaw face each other, with respect to the second electromagnetic wave receiver electrode),

wherein, under a state in which the first blade or the first jaw and the second blade or the second jaw are closed, or under a state in which the first blade or the first jaw is on a lower side of the tangential line or the second blade or the second jaw is on an upper side of the tangential line,

the first electromagnetic wave application electrode and the second electromagnetic wave application electrode face each other, and the first electromagnetic wave receiver electrode and the second electromagnetic wave receiver electrode face each other, and

wherein the medical treatment tool is configured to irradiate an electromagnetic wave between the first blade or the first jaw and the second blade or the second jaw, irradiate an electromagnetic wave on a side surface of the first blade or a side surface of the first jaw, and irradiate an electromagnetic wave on a side surface of the second blade or a side surface of the second jaw.

2. A medical treatment tool, including:

a first blade or a first jaw including a first electromagnetic wave application electrode and a first electromagnetic wave receiver electrode;

a second blade or a second jaw, which is arranged so as to be opposed to the first blade or the first jaw, and includes a second electromagnetic wave application electrode and a second electromagnetic wave receiver electrode; and

a coaxial cable including a central conductor and an external conductor,

wherein:
the first electromagnetic wave application electrode is located on an upper side of the first blade or the first jaw with respect to the first electromagnetic wave receiver electrode (or the first electromagnetic wave application electrode is located on a side farther from a line in which the first blade or the first jaw and the second blade or the second jaw face each other, with respect to the first electromagnetic wave receiver electrode) and the second electromagnetic wave receiver electrode is located on an upper side of the second blade or the second jaw with respect to the second electromagnetic wave application electrode) (or the second electromagnetic wave receiver electrode is located on a side closer to the line in which the first blade or the first jaw and the second blade or the second jaw face each other, with respect to the second electromagnetic wave application electrode), and under a state in which the first blade or the first jaw and the second blade or the second jaw are closed, the first electromagnetic wave application electrode and the second electromagnetic wave receiver electrode face each other and the first electromagnetic wave receiver electrode and the second electromagnetic wave application electrode face each other; or
the first electromagnetic wave receiver electrode is located on the upper side of the first blade or the first jaw with respect to the first electromagnetic wave application electrode (or the first electromagnetic wave receiver electrode is located on the side farther from the line in which the first blade or the first jaw and the second blade or the second jaw face each other, with respect to the first electromagnetic wave application electrode) and the second electromagnetic wave application electrode is located on the upper side of the second blade or the second jaw with respect to the second electromagnetic wave receiver electrode (the second electromagnetic wave application electrode is located on the side closer to the line in which the first blade or the first jaw and the second blade or the second jaw face each other, with respect to the second electromagnetic wave receiver electrode), and
under a state in which the first blade or the first jaw and the second blade or the second jaw are closed, or under a state in which the first blade or the first jaw is on a lower side of a tangential line or the second blade or the second jaw is on an upper side of the tangential line,
the first electromagnetic wave receiver electrode and the second electromagnetic wave application electrode face each other, and the first electromagnetic wave application electrode and the electromagnetic wave receiver electrode face each other,

wherein the central conductor is directly or indirectly connected to the first electromagnetic wave application electrode and the second electromagnetic wave application electrode, and the external conductor is directly or indirectly connected to the first electromagnetic wave receiver electrode and the second electromagnetic wave receiver electrode, and

wherein the medical treatment tool is configured to irradiate an electromagnetic wave from both the first blade or the first jaw and the second blade or the second jaw.

3. A medical treatment tool, including:

a first blade or a first jaw including a first electromagnetic wave application electrode, a third electromagnetic wave application electrode, a first electromagnetic wave receiver electrode, and a third electromagnetic wave receiver electrode;

a second blade or a second jaw, which is arranged so as to be opposed to the first blade or the first jaw, and includes a second electromagnetic wave application electrode, a fourth electromagnetic wave application electrode, a second electromagnetic wave receiver electrode, and a fourth electromagnetic wave receiver electrode; and

a coaxial cable including a central conductor and an external conductor,

wherein:
the first electromagnetic wave application electrode, the first electromagnetic wave receiver electrode, the third electromagnetic wave application electrode, and the third electromagnetic wave receiver electrode are located in the stated order from an upper side of the first blade or the first jaw (the first electromagnetic wave application electrode, the first electromagnetic wave receiver electrode, the third electromagnetic wave application electrode, and the third electromagnetic wave receiver electrode are located in the stated order from a side farther from a line in which the first blade or the first jaw and the second blade or the second jaw face each other) and the second electromagnetic wave application electrode, the second electromagnetic wave receiver electrode, the fourth electromagnetic wave application electrode, and the fourth electromagnetic wave receiver electrode are located in the stated order from an upper side of the second blade or the second jaw (the second electromagnetic wave application electrode, the second electromagnetic wave receiver electrode, the fourth electromagnetic wave application electrode, and the fourth electromagnetic wave receiver electrode are located in the stated order from a side closer to the line in which the first blade or the first jaw and the second blade or the second jaw face each other); and
under a state in which the first blade or the first jaw and the second blade or the second jaw are closed, or under a state in which the first blade or the first jaw is on a lower side of a tangential line or the second blade or the second jaw is on an upper side of the tangential line, the first electromagnetic wave application electrode and second electromagnetic wave application electrode, the first electromagnetic wave receiver electrode and second electromagnetic wave receiver electrode, the third electromagnetic wave application electrode and fourth electromagnetic wave application electrode, and the third electromagnetic wave receiver electrode and fourth electromagnetic wave receiver electrode face each other, respectively,

wherein the central conductor is directly or indirectly connected to the first electromagnetic wave application electrode, the second electromagnetic wave application electrode, the third electromagnetic wave application electrode, and the fourth electromagnetic wave application electrode, and the external conductor is directly or indirectly connected to the first electromagnetic wave receiver electrode, the second electromagnetic wave receiver electrode, the third electromagnetic wave receiver electrode, and the fourth electromagnetic wave receiver electrode, and

wherein the medical treatment tool is configured to irradiate an electromagnetic wave between the first blade or the first jaw and the second blade or the second jaw, irradiate an electromagnetic wave on a side surface of the first blade or a side surface of the first jaw, and irradiate an electromagnetic wave on a side surface of the second blade or a side surface of the second jaw.

4. A medical treatment tool, including:

a first blade or a first jaw including a first electromagnetic wave application electrode, a third electromagnetic wave application electrode, a first electromagnetic wave receiver electrode, and a third electromagnetic wave receiver electrode;

a second blade or a second jaw, which is arranged so as to be opposed to the first blade or the first jaw, and includes a second electromagnetic wave application electrode, a fourth electromagnetic wave application electrode, a second electromagnetic wave receiver electrode, and a fourth electromagnetic wave receiver electrode; and

a coaxial cable including a central conductor and an external conductor,

wherein:
the first electromagnetic wave application electrode), the first electromagnetic wave receiver electrode, the third electromagnetic wave application electrode, and the third electromagnetic wave receiver electrode are located in the stated order from an upper side of the first blade or the first jaw (the first electromagnetic wave application electrode, the first electromagnetic wave receiver electrode, the third electromagnetic wave application electrode, and the third electromagnetic wave receiver electrode are located in the stated order from a side farther from a line in which the first blade or the first jaw and the second blade or the second jaw face each other) and the second electromagnetic wave receiver electrode, the second electromagnetic wave application electrode, the fourth electromagnetic wave receiver electrode, and the fourth electromagnetic wave application electrode are located in the stated order from an upper side of the second blade or the second jaw (the second electromagnetic wave receiver electrode, the second electromagnetic wave application electrode, the fourth electromagnetic wave receiver electrode, and the fourth electromagnetic wave application electrode are located in the stated order from a side closer to the line in which the first blade or the first jaw and the second blade or the second jaw face each other); and
under a state in which the first blade or the first jaw and the second blade or the second jaw are closed, or under a state in which the first blade or the first jaw is on a lower side of a tangential line or the second blade or the second jaw is on an upper side of the tangential line, the first electromagnetic wave application electrode and second electromagnetic wave receiver electrode, the second electromagnetic wave application electrode and first electromagnetic wave receiver electrode, the third electromagnetic wave application electrode and fourth electromagnetic wave receiver electrode, and the fourth electromagnetic wave application electrode and third electromagnetic wave receiver electrode face each other, respectively,

wherein the central conductor is directly or indirectly connected to the first electromagnetic wave application electrode, the second electromagnetic wave application electrode, the third electromagnetic wave application electrode, and the fourth electromagnetic wave application electrode, and the external conductor is directly or indirectly connected to the first electromagnetic wave receiver electrode, the second electromagnetic wave receiver electrode, the third electromagnetic wave receiver electrode, and the fourth electromagnetic wave receiver electrode, and

wherein the medical treatment tool is configured to irradiate an electromagnetic wave from both the first blade or the first jaw and the second blade or the second jaw.

5. A medical treatment tool, including:

a first blade or a first jaw including a first electromagnetic wave application electrode, a first electromagnetic wave receiver electrode, a third electromagnetic wave application electrode, a third electromagnetic wave receiver electrode, a fifth electromagnetic wave application electrode, and a fifth electromagnetic wave receiver electrode;

a second blade or a second jaw, which is arranged so as to be opposed to the first blade or the first jaw, and includes a second electromagnetic wave receiver electrode, a second electromagnetic wave application electrode, a fourth electromagnetic wave receiver electrode, a fourth electromagnetic wave application electrode, a sixth electromagnetic wave receiver electrode, and a sixth electromagnetic wave application electrode,
the first electromagnetic wave application electrode, the first electromagnetic wave receiver electrode, the third electromagnetic wave application electrode, the third electromagnetic wave receiver electrode, the fifth electromagnetic wave application electrode, and the fifth electromagnetic wave receiver electrode being arranged in the stated order from a distal end side of the first blade or the first jaw, and the second electromagnetic wave receiver electrode, the second electromagnetic wave application electrode, the fourth electromagnetic wave receiver electrode, the fourth electromagnetic wave application electrode, the sixth electromagnetic wave receiver electrode, and the sixth electromagnetic wave application electrode being arranged in the stated order from a distal end side of the second blade or the second jaw; and

a coaxial cable including a central conductor and an external conductor,

wherein the central conductor is directly or indirectly connected to the first electromagnetic wave application electrode, the second electromagnetic wave application electrode, the third electromagnetic wave application electrode, the fourth electromagnetic wave application electrode, the fifth electromagnetic wave application electrode, and the sixth electromagnetic wave application electrode, and the external conductor is directly or indirectly connected to the first electromagnetic wave receiver electrode, the second electromagnetic wave receiver electrode, the third electromagnetic wave receiver electrode, the fourth electromagnetic wave receiver electrode, the fifth electromagnetic wave receiver electrode, and the sixth electromagnetic wave receiver electrode,

wherein, under a state in which the first blade or the first jaw and the second blade or the second jaw are closed, the first electromagnetic wave application electrode and second electromagnetic wave receiver electrode, the first electromagnetic wave receiver electrode and second electromagnetic wave application electrode, the third electromagnetic wave application electrode and fourth electromagnetic wave receiver electrode, the third electromagnetic wave receiver electrode and fourth fifth electromagnetic wave application electrode, the electromagnetic wave application electrode and sixth electromagnetic wave receiver electrode, and the fifth electromagnetic wave receiver electrode and sixth electromagnetic wave application electrode face each other, respectively, and

wherein the medical treatment tool is configured to irradiate an electromagnetic wave between the first blade or the first jaw and the second blade or the second jaw, irradiate an electromagnetic wave on a side surface of the first blade or a side surface of the first jaw, and irradiate an electromagnetic wave on a side surface of the second blade or a side surface of the second jaw.

6. The medical treatment tool according to the above-mentioned item 1 or 2, wherein a part or all of the first electromagnetic wave application electrode and the first electromagnetic wave receiver electrode are built or buried in the first blade or the first jaw, and a part or all of the second electromagnetic wave application electrode and the second electromagnetic wave receiver electrode are built or buried in the second blade or the second jaw.

7. The medical treatment tool according to the above-mentioned item 3 or 4, wherein a part or all of the first electromagnetic wave application electrode, the first electromagnetic wave receiver electrode, the third electromagnetic wave application electrode, and the third electromagnetic wave receiver electrode are built or buried in the first blade or the first jaw, and a part or all of the second electromagnetic wave application electrode, the second electromagnetic wave receiver electrode, the fourth electromagnetic wave application electrode and the fourth electromagnetic wave receiver electrode are built or buried in the second blade or the second jaw.

8. The medical treatment tool according to the above-mentioned item 1 or 2, wherein, in the first blade or the first jaw, the first electromagnetic wave application electrode and the first electromagnetic wave receiver electrode are installed through intermediation of an insulator or a dielectric body, and in the second blade or the second jaw, the second electromagnetic wave application electrode and the second electromagnetic wave receiver electrode are installed through intermediation of an insulator or a dielectric body.

9. The medical treatment tool according to the above-mentioned item 3 or 4, wherein, in the first blade or the first jaw, the first electromagnetic wave application electrode, the first electromagnetic wave receiver electrode, the third electromagnetic wave application electrode, and the third electromagnetic wave receiver electrode are installed through intermediation of an insulator or a dielectric body, and in the second blade or the second jaw, the second electromagnetic wave application electrode, the second electromagnetic wave receiver electrode, the fourth electromagnetic wave application electrode, and the fourth electromagnetic wave receiver electrode are installed through intermediation of an insulator or a dielectric body.

10. The medical treatment tool according to the above-mentioned item 1, 3, or 5, further including a switch,

wherein the central conductor is directly or indirectly connected to the first electromagnetic wave application electrode, the second electromagnetic wave application electrode, the third electromagnetic wave application electrode, the fourth electromagnetic wave application electrode, the fifth electromagnetic wave application electrode, and the sixth electromagnetic wave application electrode through intermediation of the switch, and the irradiation of the electromagnetic wave includes any one or more of the following flows:

(1) the electromagnetic wave is irradiated between the first blade or the first jaw and the second blade or the second jaw;
(2) the electromagnetic wave is irradiated on the side surface of the first blade or the side surface of the first jaw; and
(3) the electromagnetic wave is irradiated on the side surface of the second blade or the side surface of the second jaw.

11. The medical treatment tool according to any one of the above-mentioned items 1 to 10, wherein the insulator or the derivative is a fluorine resin or quartz.

12. The medical treatment tool according to any one of the above-mentioned items 1 to 11, wherein the electromagnetic wave is a microwave.

13. The medical treatment tool according to any one of the above-mentioned items 1 to 12, further including a grip or a trigger directly or indirectly connected to the first blade or the first jaw and/or the second blade or the second jaw,

wherein opening/closing of the first blade or the first jaw and/or the second blade or the second jaw is performed through a movement of the trigger or the grip.

14. The medical treatment tool according to any one of the above-mentioned items 1 to 13, further including:

a wrist joint movement portion connected to a base end side of the first blade or the first jaw and the second blade or the second jaw;

a shaft connected to a base end side of the wrist joint movement portion; and

a power connecting portion connected to a base end side of the shaft,

wherein the medical treatment tool is configured to automatically perform, through power of the power connecting portion, an action of the wrist joint portion, an opening and closing operation of the blades or the jaws, a rotation of the shaft, and a back-and-forth movement of the shaft in a longitudinal direction.

15. An electromagnetic wave medical system, including:

the medical treatment tool of any one of the above-mentioned items 1 to 14;

an electromagnetic wave generating apparatus configured to supply an electromagnetic wave to the treatment tool;

a drive unit configured to operate the treatment tool;

a monitor configured to present an image of the treatment tool; and

a control unit configured to control the monitor, the medical treatment tool, the electromagnetic wave generating apparatus, and the drive unit.

16. An organic material coagulation method, including coagulating an organic material through use of the medical treatment tool of any one of the above-mentioned items 1 to 14 or the electromagnetic wave medical system of item 12.

### Advantageous Effects of Invention

The medical treatment tool according to the present invention has any one or more of the following effects.

(1) When tissue is subjected to incision/cutting between the blade pair, a microwave can be applied to the tissue to dielectrically heat the tissue.
(2) The side surface of the blade can dielectrically heat the tissue part brought into abutment against the side surface of the blade.
(3) The tissue part in which the tissue is nipped between blades and the tissue part brought into abutment against the side surface of the blade can be dielectrically heated simultaneously or sequentially.
(4) The tissue can be dielectrically heated only by the side surface of the blade, and hence coagulation can be performed only by one blade or one jaw.

### BRIEF DESCRIPTION OF DRAWINGS

FIG. 1 is a diagram of blade pairs of a first patter to a fourth pattern to of a medical treatment tool 1 of the present invention (Each arrow means a flow of a microwave. In each of the first pattern and the second patterns, an electromagnetic wave is irradiated between a first blade or a first jaw and a second blade or a second jaw, an electromagnetic wave is irradiated on a side surface of the first blade or a side surface of the first jaw, and an electromagnetic wave is irradiated on a side surface of the second blade or a side surface of the second jaw).

FIG. 2 is a diagram of blade pairs of a fifth pattern and a sixth pattern of the medical treatment tool 1 according to the present invention (Each arrow means a flow of a microwave. In the sixth pattern, an electromagnetic wave is irradiated between the first blade or the first jaw and the second blade or the second jaw, an electromagnetic wave is irradiated on the side surface of the first blade or the side surface of the first jaw, and an electromagnetic wave is irradiated on the side surface of the second blade or the side surface of the second jaw).

FIG. 3 is a diagram of differences between the medical treatment tool 1 according to the present invention and a related-art medical treatment tool.

FIG. 4 a view of a distal end of the first pattern of the medical treatment tool 1 according to the present invention.

FIG. 5 is a view of a distal end of the fourth pattern of the medical treatment tool 1 according to the present invention.

FIG. 6 is a diagram of a microwave circuit (dielectric heating circuit) of the first pattern of the medical treatment tool 1 according to the present invention.

FIG. 7 is an explanatory view of electric field strengths between the blades.

FIG. 8 is a view of a scissors structure and definitions of terms.

FIG. 9 is a schematic diagram of an electromagnetic wave medical system.

FIG. 10 is a view of a medical treatment tool capable of automatically performing an action of a wrist joint portion, an opening or closing operation of the blades or the jaws, a rotation of a shaft, and a movement of the shaft in the longitudinal direction.

FIG. 11 is a view of the medical treatment tool 1 according to the present invention, which is capable of dielectrically heating a living tissue (7) brought into contact not only with blade tips but also with the side surfaces of the blades.

FIG. 12 is an overall view of a seventh pattern of the medical treatment tool 1 according to the present invention (the arrows indicate flows of an electromagnetic wave).

### DESCRIPTION OF EMBODIMENTS

Hereinafter, the present invention is described with reference to the drawings. However, the present invention is not limited to a medical treatment tool illustrated in the drawings.

(Medical Treatment Tool)

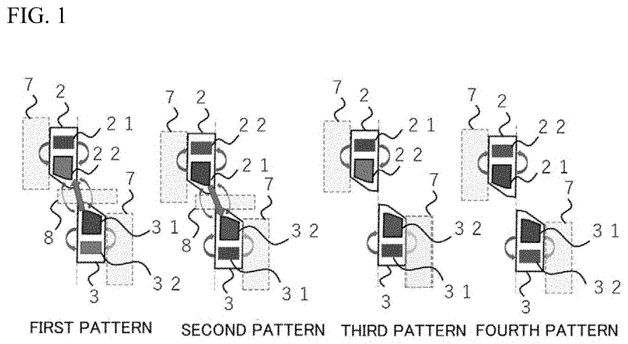

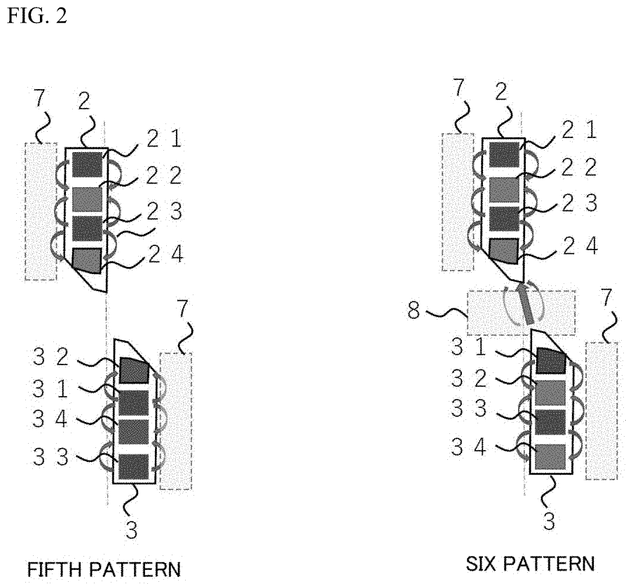

A medical treatment tool 1 according to the present invention relates to a microwave heating tool (in particular, microwave heating surgical tool) capable of electromagnetic wave (in particular, microwave) heating from side surfaces of a blade pair or a jaw pair (see FIG. 1 and FIG. 2).

The medical treatment tool 1 according to the present invention has six kinds of patterns (aspects) as illustrated in FIG. 1 and FIG. 2.

The electromagnetic wave in the present invention refers to a microwave having a frequency of from 3×10¹⁹ Hz to 300 kHz, preferably, a frequency of from 300 MHz to 300 GHz.

(Medical Treatment Tool According to the Present Invention)

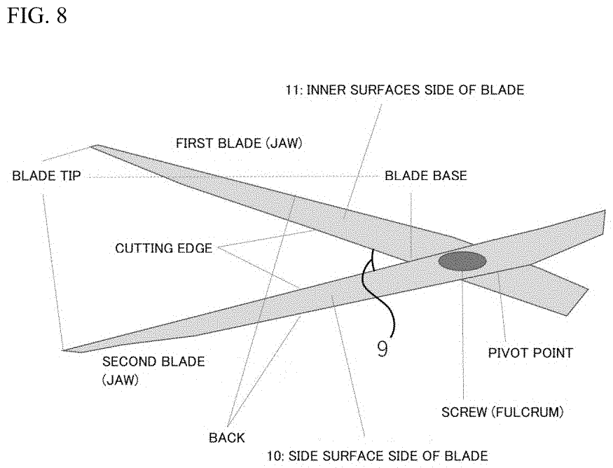

As illustrated in FIG. 1 and FIG. 2, the medical treatment tool 1 according to the present invention is a so-called scissors-type medical treatment tool, and is capable of heating an organic material {tissue (living tissue)} through use of an electromagnetic wave (in particular, microwave) from the side surfaces of the blades (blade pair), which is a feature of the electromagnetic wave (in particular, microwave) heating of a forceps/tweezers type. Specifically, it is possible to refer to a “living tissue 7 subjected to microwave heating from the side surfaces of the blades” of FIG. 1 and FIG. 2. In all the figures including FIG. 1 and FIG. 2, the upper half is referred to as “upper side” and the lower half is referred to as “lower side” when the figures are viewed from the front. FIG. 8 can be referred to for a scissors structure and terms used for description of examples.

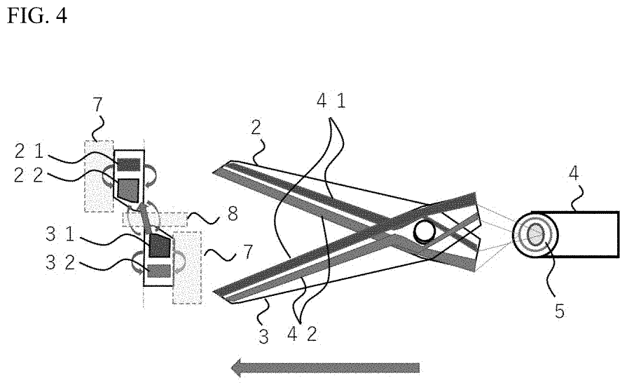

A “distal end” as used herein means a distal end side of a blade or a jaw (see FIG. 4; a direction (longitudinal axial direction (longitudinal direction)) indicated by the arrow is the distal end side, and the opposite direction is a proximal side (base end side)).

The organic material means not only the tissue (living tissue) but also general compounds containing carbon.

The medical treatment tool 1 according to the present invention continuously or simultaneously performs treatment through tissue fixation and substantially no-bleeding incision in a target part or a peripheral area of pathology, to thereby be able to improve reliability, swiftness, and safety of the treatment and alleviate a burden on a patient and an operator (doctor). More specifically, the living tissue can be subjected to tissue fixation through the electromagnetic wave (in particular, microwave) heating in virtually all surface spaces and other spaces in which the blades and the living tissue are brought into contact with each other and at all time slots before cutting, during the cutting, and after the cutting. This enables swift treatment and continuous treatment.

In a case of a related-art medical treatment tool with an antenna installed on only one side, a coagulation area has been smaller on a side opposite to the side on which the antenna is installed. However, when the medical treatment tool according to the present invention is used, for example, a living tissue 8 subjected to the microwave heating from blade tips of the blade pair can be reliably hemostatically incised. In particular, in surgical treatment of cancer, it is possible to prevent leakage of, for example, lymph, and hence there is an effect of preventing metastasis due to dissemination and scattering of cancer cells.

In addition, through use of the medical treatment tool 1 according to the present invention, the operator can subject any wide surface to the tissue fixation on the side surfaces of the blades through microwave irradiation from the side surfaces of the blade pair. In a case of excision of a liver having tissue in contact with the side surfaces of blades, not only tissue at cutting edges of the blades but also cross sections on the side surfaces are subjected to the tissue fixation, and a fixed local area can be incised immediately or simultaneously. Thus, it is possible to completely perform hemostatic incision without changing the medical treatment tool. In addition, the tissue can be nipped from both sides, and most of parts irradiated with the microwave can be used in contact with tissue surfaces to be incised, and it is possible to efficiently perform coagulation and hemostasis without having the microwave absorbed by a surrounding environment, for example, into body fluid.

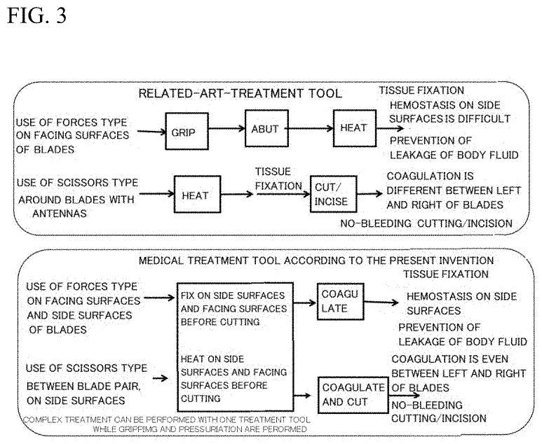

More details can be described with reference to a functional diagram illustrated in FIG. 3. As illustrated in FIG. 3, in the medical treatment tool 1 according to the present invention, both forceps-type/tweezers-type treatment and scissors-type treatment can be executed through use of one treatment tool, and hence it is possible to continuously or simultaneously perform treatment through the tissue fixation and the no-bleeding incision in a peripheral area of pathology, to thereby improve the reliability, swiftness, and safety of the treatment and alleviate a burden on a patient and an operator.

(First Pattern and Second Pattern)

The first pattern and the second pattern of the medical treatment tool 1 according to the present invention include at least the following configurations.

First Pattern (FIG. 4)

A first blade or a first jaw including a first electromagnetic wave (microwave) application electrode and a first electromagnetic wave (microwave) receiver electrode.

A second blade or a second jaw, which is arranged so as to be opposed to the first blade or the first jaw, and includes a second electromagnetic wave (microwave) application electrode and a second electromagnetic wave (microwave) receiver electrode.

A coaxial cable including a central conductor and an external conductor.

The central conductor is directly or indirectly connected to the first electromagnetic wave (microwave) application electrode and the second electromagnetic wave (microwave) application electrode, and the external conductor is directly or indirectly connected to the first electromagnetic wave (microwave) receiver electrode and the second electromagnetic wave (microwave) receiver electrode.

In addition, the first electromagnetic wave (microwave) application electrode is located on the upper side of the first blade or the first jaw with respect to the first electromagnetic wave (microwave) receiver electrode, and the second electromagnetic wave (microwave) application electrode is located on the upper side of the second or the second jaw with respect to the second electromagnetic wave (microwave) receiver electrode. Under a state in which the first blade or the first jaw and the second blade or the second jaw are closed, the first electromagnetic wave (microwave) application electrode and the second electromagnetic wave (microwave) application electrode face each other, and the first electromagnetic wave (microwave) receiver electrode and the second electromagnetic wave (microwave) receiver electrode face each other.

The first blade and the second blade, and the first jaw and the second jaw, are described separately for the sake of the description of the invention, but those descriptions are changeable. The same applies to the other patterns.

Second Pattern

A first blade or a first jaw including an first electromagnetic wave (microwave) application electrode and a first electromagnetic wave (microwave) receiver electrode.

A second blade or a second jaw, which is arranged so as to be opposed to the first blade or the first jaw, and includes a second electromagnetic wave (microwave) application electrode and a second electromagnetic wave (microwave) receiver electrode.

A coaxial cable including a central conductor and an external conductor.

The central conductor is directly or indirectly connected to the first electromagnetic wave (microwave) application electrode and the second electromagnetic wave (microwave) application electrode, and the external conductor is directly or indirectly connected to the first electromagnetic wave (microwave) receiver electrode and the second electromagnetic wave (microwave) receiver electrode.

In addition, the first electromagnetic wave (microwave) application electrode is located on the upper side of the first blade or the first jaw with respect to the first electromagnetic wave (microwave) receiver electrode, and the second electromagnetic wave (microwave) receiver electrode is located on the upper side of the second or the second jaw with respect to the second electromagnetic wave (microwave) application electrode. Under a state in which the first blade or the first jaw and the second blade or the second jaw are closed, the first electromagnetic wave (microwave) application electrode and the second electromagnetic wave (microwave) application electrode face each other, and the first electromagnetic wave (microwave) receiver electrode and the second electromagnetic wave (microwave) receiver electrode face each other.

In the first pattern (see FIG. 4) and the second pattern of the medical treatment tool 1 according to the present invention, it is not only possible to perform the tissue fixation on a wide surface through use of the side surfaces of the blades or the jaws based on the electromagnetic wave (microwave) heating from the side surfaces of the blades or the jaws, but also possible to reliably hemostatically incise the living tissue 8 subjected to the electromagnetic wave (microwave) heating from the blade tips of the blade pair or the jaw pair.

(Third Pattern and Fourth Pattern)

The third pattern and the fourth patter of the medical treatment tool 1 according to the present invention include at least the following configurations.

Third Pattern

A first blade or a first jaw including a first electromagnetic wave (microwave) application electrode and a first electromagnetic wave (microwave) receiver electrode.

A second blade or a second jaw, which is arranged so as to be opposed to the first blade or the first jaw, and includes a second electromagnetic wave (microwave) application electrode and a second electromagnetic wave (microwave) receiver electrode.

A coaxial cable including a central conductor and an external conductor.

The central conductor is directly or indirectly connected to the first electromagnetic wave (microwave) application electrode and the second electromagnetic wave (microwave) application electrode, and the external conductor is directly or indirectly connected to the first electromagnetic wave (microwave) receiver electrode and the second electromagnetic wave (microwave) receiver electrode.

In addition, the first electromagnetic wave (microwave) application electrode is located on the upper side of the first blade or the first jaw with respect to the first electromagnetic wave (microwave) receiver electrode, and the second electromagnetic wave (microwave) receiver electrode is located on the upper side of the second or the second jaw with respect to the second electromagnetic wave (microwave) application electrode. Under a state in which the first blade or the first jaw and the second blade or the second jaw are closed, the first electromagnetic wave (microwave) application electrode and the second electromagnetic wave (microwave) receiver electrode face each other, and the first electromagnetic wave (microwave) receiver electrode and the second electromagnetic wave (microwave) application electrode face each other.

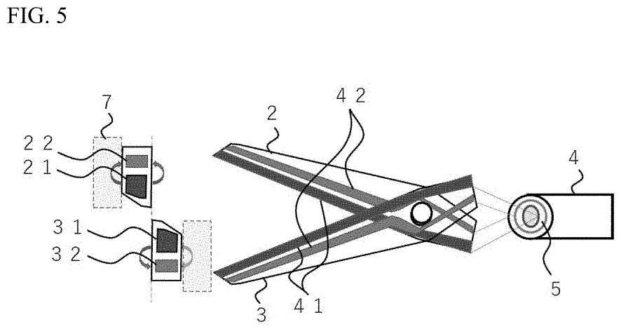

Fourth Pattern (FIG. 5)

A first blade or a first jaw including a first electromagnetic wave (microwave) application electrode and a first electromagnetic wave (microwave) receiver electrode.

A second blade or a second jaw, which is arranged so as to be opposed to the first blade or the first jaw, and includes a second electromagnetic wave (microwave) application electrode and a second electromagnetic wave (microwave) receiver electrode.

A coaxial cable including a central conductor and an external conductor.

The central conductor is directly or indirectly connected to the first electromagnetic wave (microwave) application electrode and the second electromagnetic wave (microwave) application electrode, and the external conductor is directly or indirectly connected to the first electromagnetic wave (microwave) receiver electrode and the second electromagnetic wave (microwave) receiver electrode.

In addition, the first electromagnetic wave (microwave) receiver electrode is located on the upper side of the first blade or the first jaw with respect to the first electromagnetic wave (microwave) application electrode, and the second electromagnetic wave (microwave) application electrode is located on the upper side of a second electrode with respect to the second electromagnetic wave (microwave) receiver electrode. Under a state in which the first blade or the first jaw and the second blade or the second jaw are closed, the first electromagnetic wave (microwave) receiver electrode and the second electromagnetic wave (microwave) application electrode face each other, and the first electromagnetic wave (microwave) application electrode and the second electromagnetic wave (microwave) receiver electrode face each other.

In the third pattern and the fourth pattern (see FIG. 5) of the medical treatment tool 1 according to the present invention, it is possible to perform the tissue fixation on a wide surface through use of the side surfaces of the blades or the jaws based on the electromagnetic wave (microwave) heating from the side surfaces of the blades or the jaws.

(Fifth Pattern and Sixth Pattern)

The fifth pattern and the six pattern of the medical treatment tool 1 according to the present invention include at least the following configurations.

Fifth Pattern (FIG. 2)

A first blade or a first jaw including a first electromagnetic wave (microwave) application electrode, a third electromagnetic wave (microwave) application electrode, a first electromagnetic wave (microwave) receiver electrode, and a third electromagnetic wave (microwave) receiver electrode.

A second blade or a second jaw, which is arranged so as to be opposed to the first blade or the first jaw, and includes a second electromagnetic wave (microwave) application electrode, a fourth electromagnetic wave (microwave) application electrode, a second electromagnetic wave (microwave) receiver electrode, and a fourth electromagnetic wave (microwave) receiver electrode.

A coaxial cable including a central conductor and an external conductor.

The first electromagnetic wave (microwave) application electrode, the first electromagnetic wave (microwave) receiver electrode, the third electromagnetic wave (microwave) application electrode, and the third electromagnetic wave (microwave) receiver electrode are located in the stated order from the upper side of the first blade or the first jaw, and the second electromagnetic wave (microwave) receiver electrode, the second electromagnetic wave (microwave) application electrode, the fourth electromagnetic wave (microwave) receiver electrode, and the fourth electromagnetic wave (microwave) application electrode are located in the stated order from the upper side of the second blade or the second jaw.

The central conductor is directly or indirectly connected to the first electromagnetic wave (microwave) application electrode, the second electromagnetic wave (microwave) application electrode, the third electromagnetic wave (microwave) application electrode, and the fourth electromagnetic wave (microwave) application electrode, and the external conductor is directly or indirectly connected to the first electromagnetic wave (microwave) receiver electrode, the second electromagnetic wave (microwave) receiver electrode, the third electromagnetic wave (microwave) receiver electrode, and the fourth electromagnetic wave (microwave) receiver electrode.

In addition, under the state in which the first blade or the first jaw and the second blade or the second jaw are closed, the first electromagnetic wave (microwave) application electrode and second electromagnetic wave (microwave) receiver electrode, the second electromagnetic wave (microwave) application electrode and first electromagnetic wave (microwave) receiver electrode, the third electromagnetic wave (microwave) application electrode and fourth electromagnetic wave (microwave) receiver electrode, and the fourth electromagnetic wave (microwave) application electrode and third electromagnetic wave (microwave) receiver electrode face each other, respectively.

In the fifth pattern of the medical treatment tool 1 according to the present invention, it is possible to perform the tissue fixation on a wide surface through use of the side surfaces of the blades or the jaws based on the electromagnetic wave (microwave) heating from the side surfaces of the blades or the jaws.

Sixth Pattern (FIG. 2)

A first blade or a first jaw including a first electromagnetic wave (microwave) application electrode, a third electromagnetic wave (microwave) application electrode, a first electromagnetic wave (microwave) receiver electrode, and a third electromagnetic wave (microwave) receiver electrode.

A second blade or a second jaw, which is arranged so as to be opposed to the first blade or the first jaw, and includes a second electromagnetic wave (microwave) application electrode, a fourth electromagnetic wave (microwave) application electrode, a second electromagnetic wave (microwave) receiver electrode, and a fourth electromagnetic wave (microwave) receiver electrode.

A coaxial cable including a central conductor and an external conductor.

The first electromagnetic wave (microwave) application electrode, the first electromagnetic wave (microwave) receiver electrode, the third electromagnetic wave (microwave) application electrode, and the third electromagnetic wave (microwave) receiver electrode are located in the stated order from the upper side of the first blade or the first jaw, and the second electromagnetic wave (microwave) application electrode, the second electromagnetic wave (microwave) receiver electrode, the fourth electromagnetic wave (microwave) application electrode, and the fourth electromagnetic wave (microwave) receiver electrode are located in the stated order from the upper side of the second blade or the second jaw.

The central conductor is directly or indirectly connected to the first electromagnetic wave (microwave) application electrode, the second electromagnetic wave (microwave) application electrode, the third electromagnetic wave (microwave) application electrode, and the fourth electromagnetic wave (microwave) application electrode, and the external conductor is directly or indirectly connected to the first electromagnetic wave (microwave) receiver electrode, the second electromagnetic wave (microwave) receiver electrode, the third electromagnetic wave (microwave) receiver electrode, and the fourth electromagnetic wave (microwave) receiver electrode.

Under the state in which the first blade or the first jaw and the second blade or the second jaw are closed, the first electromagnetic wave (microwave) application electrode and second electromagnetic wave (microwave) application electrode, the first electromagnetic wave (microwave) receiver electrode and second electromagnetic wave (microwave) receiver electrode, the third electromagnetic wave (microwave) application electrode and fourth electromagnetic wave (microwave) application electrode, and the third electromagnetic wave (microwave) receiver electrode and fourth electromagnetic wave (microwave) receiver electrode face each other, respectively.

In the sixth pattern (see FIG. 2) of the medical treatment tool 1 according to the present invention, it is possible to reliably hemostatically incise the living tissue 7 subjected to the electromagnetic wave (microwave) heating from the blade tips of the blade pair or the jaw pair, and simultaneously possible to perform the tissue fixation on a wide surface through use of the side surfaces of the blades or the jaws based on the electromagnetic wave (microwave) heating from the side surfaces of the blades or the jaws. When the tissue is fixed on a wide surface in advance on the side surfaces of the blades or the jaws and then incised or cut, it is possible to perform treatment by further improving a degree of reliability in bleeding prevention.

(Seventh Pattern)

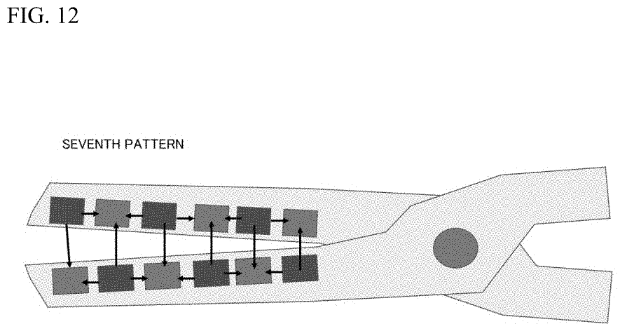

The seventh pattern (FIG. 12) of the medical treatment tool 1 according to the present invention include at least the following configurations.

A first blade or a first jaw including a first electromagnetic wave application electrode, a first electromagnetic wave receiver electrode, a third electromagnetic wave application electrode, a third electromagnetic wave receiver electrode, a fifth electromagnetic wave application electrode, and a fifth electromagnetic wave receiver electrode.

A second blade or a second jaw, which is arranged so as to be opposed to the first blade or the first jaw, and includes a second electromagnetic wave receiver electrode, a second electromagnetic wave application electrode, a fourth electromagnetic wave receiver electrode, a fourth electromagnetic wave application electrode, a sixth electromagnetic wave receiver electrode, and a sixth electromagnetic wave application electrode.

The first electromagnetic wave application electrode, the first electromagnetic wave receiver electrode, the third electromagnetic wave application electrode, the third electromagnetic wave receiver electrode, the fifth electromagnetic wave application electrode, and the fifth electromagnetic wave receiver electrode are located in the stated order from the distal end side of the first blade or the first jaw, and the second electromagnetic wave receiver electrode, the second electromagnetic wave application electrode, the fourth electromagnetic wave receiver electrode, the fourth electromagnetic wave application electrode, the sixth electromagnetic wave receiver electrode, and the sixth electromagnetic wave application electrode are located in the stated order from the distal end side of the second blade or the second jaw.

A coaxial cable including a central conductor and an external conductor.

The central conductor is directly or indirectly connected to the first electromagnetic wave application electrode, the second electromagnetic wave application electrode, the third electromagnetic wave application electrode, the fourth electromagnetic wave application electrode, the fifth electromagnetic wave application electrode, and the sixth electromagnetic wave application electrode, and the external conductor is directly or indirectly connected to the first electromagnetic wave receiver electrode, the second electromagnetic wave receiver electrode, the third electromagnetic wave receiver electrode, the fourth electromagnetic wave receiver electrode, the fifth electromagnetic wave receiver electrode, and the sixth electromagnetic wave receiver electrode.

Under the state in which the first blade or the first jaw and the second blade or the second jaw are closed, the first electromagnetic wave application electrode and second electromagnetic wave receiver electrode, the first electromagnetic wave receiver electrode and second electromagnetic wave application electrode, the third electromagnetic wave application electrode and fourth electromagnetic wave receiver electrode, the third electromagnetic wave receiver electrode and fourth electromagnetic wave application electrode, the fifth electromagnetic wave application electrode and sixth electromagnetic wave receiver electrode, and the fifth electromagnetic wave receiver electrode and sixth electromagnetic wave application electrode face each other, respectively.

An electromagnetic wave is irradiated between the first blade or the first jaw and the second blade or the second jaw, an electromagnetic wave is irradiated on the side surface of the first blade or the side surface of the first jaw, and an electromagnetic wave is irradiated on the side surface of the second blade or the side surface of the second jaw.

The medical treatment tool 1 according to the present invention is not particularly limited as long as the blade pair being the first blade 2 and the second blade 3 are movable so as to be able to cut and incise living tissue. For example, both the first blade 2 and the second blade 3 may be movable blades, or one thereof may be a fixed blade.

The medical treatment tool 1 according to the present invention can further include a grip and a trigger directly or indirectly connected to the first blade or the first jaw and/or the second blade or the second jaw. The user or an operator (doctor) holds the grip with his or her palm and moves (pulls) the trigger to a tail end side with his or/her index finger, to thereby be able to open or close the first blade or the first jaw and the second blade or the second jaw.

(Heat Microwave)

A microwave to be transmitted to a microwave transmission coaxial cable 4 of the medical treatment instrument 1 according to the present invention is not particularly limited, but is a microwave having a frequency of from 300 MHz to 300 GHz (wavelength: from 1 m to 1 mm), preferably from 0.9 GHz to 5 GHz. A transmission method can easily be achieved by any publicly known method, for example, a method of connecting the medical treatment instrument 1 to a publicly known microwave oscillator for oscillating a microwave or a method of building the oscillator in the medical treatment instrument 1.

Power used in the present invention is from 0.1 W to 200 W, preferably from 1.0 W to 80 W.

(Coaxial Cable)

The coaxial cable 4 used in the present invention is formed of, for example, a central conductor 41, a shield tube, and an earth pipe or a braided copper wire. The central conductor 41 is an electric conductor made of copper. The shield tube is an insulator or a dielectric body (which is made of, for example, Teflon (trademark), polyethylene, or quartz) for covering the central conductor 41. The earth pipe or the braided copper wire is an external conductor 42 (electric conductor) made of, for example, copper, stainless steel, or brass.

The coaxial cable 4 may be a publicly known semi-rigid coaxial cable.

The central conductor 41 may have a shape in which the distal end is branched off into two central conductors, and the first microwave application electrode and the second microwave application electrode can be directly or indirectly connected to the two branched-off central conductors. The central conductor 41 may also have a shape in which the distal end is branched off into four central conductors, and the first microwave application electrode, the second microwave application electrode, the third microwave application electrode, and the fourth microwave application electrode can be directly or indirectly connected to the four branched-off central conductors.

(Electrode)

The first, the second, the third, and the fourth microwave application electrodes of the present invention are not particularly limited as long as the microwave application electrodes are made of a material that enables supply of a microwave. A conductive material, such as silver, copper, gold, iron, titanium, stainless steel, phosphor bronze, or brass, may be widely used. Suitable examples thereof include silver, copper, gold, stainless steel, and brass.

The first, the second, the third, and the fourth microwave reception electrodes of the present invention are not particularly limited as long as the microwave reception electrodes are made of a material that enables reception of a microwave. A conductive material, such as silver, copper, gold, iron, titanium, stainless steel, phosphor bronze, or brass, may be widely used. Suitable examples thereof include silver, copper, gold, stainless steel, and brass.

A shape of the electrode is not particularly limited. Examples of the shape of the electrode include a circular cone, a triangular pyramid, a quadrangular pyramid, a column, a quadrangular prism, a triangular prism, a sphere, a cube, and a cuboid.

A specific shape (blade shape) of the microwave application electrode in the present invention is a quadrangular prism, and a specific shape of the microwave receiver electrode is a quadrangular prism.

An insulating dielectric body (for example, tetrafluoroethylene resin, polyethylene, polypropypene, polystyrene, polyisoputylene, polybudene, butadiene, PEEK resin, Teflon (trademark), or ceramics) may be applied between the electrodes so as to maintain a dielectric withstanding voltage between the electrodes.

In particular, the medical treatment tool 1 according to the present invention is preferred to employ a fluorine resin or quartz (quartz SiO₂) having a small ε·tan δ product so as to allow an electromagnetic wave to pass therethrough without being heated thereby.

(Blade)

The blade may be made of any material. However, for example, metal or ceramics can be used therefor.

In addition, the blade may be partially or entirely provided with a coating that prevents coagulated tissue from easily adhering thereto when the blade is brought into contact with the tissue. The blade is coated with, for example, a Teflon-based member or a silicon-based member. Thus, high water repellency is obtained to remove water-soluble components that easily cause resistance heating, and the coating functions as the above-mentioned insulating dielectric body. At the same time, the coagulation and cutting processes can be continuously performed without causing the adhesion of the tissue that has been coagulated.

(Microwave Splitter)

The medical treatment tool according to the present invention may include a microwave splitter as the requirement arises. For example, a microwave splitter known per se can be employed as long as the microwave splitter can split a microwave transmitted from a microwave transmission coaxial cable into two central conductors (first central conductor and second central conductor). Examples thereof can include a Wilkinson power divider, a 3-dB coupler splitter circuit, a rat-race splitter circuit, and a 90-degree hybrid splitter circuit.

In addition, there may be installed an apparatus (circuit) required for matching an impedance (microwave oscillation impedance) of the microwave transmission coaxial cable with impedances of a first coaxial cable and a second coaxial cable. Such a circuit can perform impedance matching by including a coil and a plurality of variable capacitors and adjusting an electrostatic capacitance of the variable capacitor, or by including, for example, a stub and a sleeve having variable lengths and adjusting their lengths.

(Switch)

The medical treatment tool according to the present invention may include a switch.

When the central conductor is directly or indirectly connected to the first electromagnetic wave application electrode, the second electromagnetic wave application electrode, the third electromagnetic wave application electrode, the fourth electromagnetic wave application electrode, the fifth electromagnetic wave application electrode, and the sixth electromagnetic wave application electrode via the switch, any one or more of the following flows of the electromagnetic wave can be achieved.

(1) An electromagnetic wave is irradiated between the first blade or the first jaw and the second blade or the second jaw.
(2) An electromagnetic wave is irradiated on the side surface of the first blade or the side surface of the first jaw.
(3) An electromagnetic wave is irradiated on the side surface of the second blade or the side surface of the second jaw.

That is, the switch enables selection of both or one of side surface heating and inter-blade heating. Thus, an effect of preventing the tissue fixation to an excessive area can be obtained, and normal tissue can be protected.

(Automatic Operation of Medical Treatment Tool)

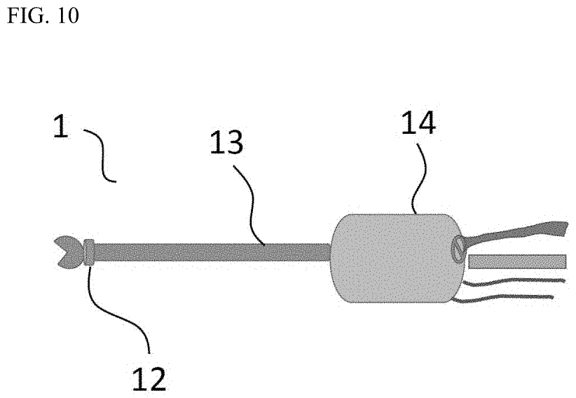

The medical treatment tool 1 according to the present invention further includes a wrist joint movement portion 12 connected to the base end side of the first blade or the first jaw 2 and the second blade or the second jaw 3, a shaft 13 connected to the base end side of the wrist joint movement portion 12, and a power connecting portion 14 connected to the base end side of the shaft 13 (see FIG. 10). It is preferred that the shaft 13 can be bent.

Through the power of the power connecting portion 14, it is possible to automatically perform an action of the wrist joint portion, an opening and closing operation of the blades or the jaws, a rotation of the shaft 13, and a back-and-forth movement of the shaft 13 in the longitudinal direction.

With this configuration, the medical treatment tool 1 according to the present invention can be used as a microwave energy surgical robot hand.

(Electromagnetic Wave Medical System)

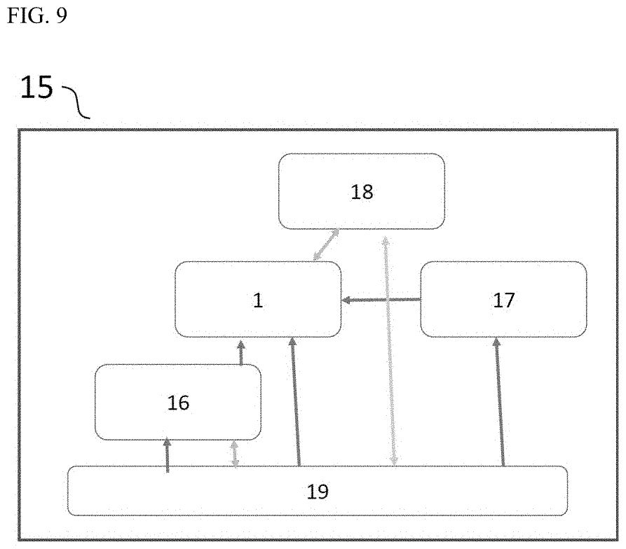

An electromagnetic wave medical system 15 according to the present invention (see FIG. 9) includes the medical treatment tool or automatically operable medical treatment tool 1, an electromagnetic wave generating apparatus 16 configured to supply the electromagnetic wave to the medical treatment tool 1, a drive unit 17 configured to operate the medical treatment tool 1 {more specifically, the unit operates the medical treatment tool 1 through the power connecting portion 14}, a monitor 18 configured to present an image of the medical treatment tool 1 (which is not limited to the image of the medical treatment tool, and also includes, for example, a position of a tumor, a treatment range, and a position of a drug), and a control unit 19 configured to control the monitor 18, the medical treatment tool 1, the electromagnetic wave generating apparatus 16, and the drive unit 17. In addition, an amplifier configured to amplify an electromagnetic wave may be included as the requirement arises.

With the presence of the control unit 19, the electromagnetic wave medical system 15 according to the present invention performs an operation required for surgery by operating the medical treatment tool in accordance with an instruction (for example, treatment program) set by an operator (for example, doctor) of this system.

(Blade Manufacturing Method)

A method of manufacturing the blades in the present invention is not particularly limited as long as it is possible to carry out the electromagnetic wave (microwave) heating from the side surfaces of the blades as described above. Examples thereof can include a method of forming an electrode on a surface of an insulating dielectric body and a method of insert molding a bulk electrode in an insulating dielectric body.

More specifically, when a part or all of each electrode is built in the blade, the following manufacturing method can be exemplified by the following.

(1) Each electrode is subjected to insert molding through use of a ceramic injection mold.

(2) A metal injection mold and a ceramic injection mold are used in combination as the requirement arises.

(3) Each electrode is molded through use of a direct bonded cupper substrate.

The blade is molded through use of a ceramic injection mold or by metal processing.

When each electrode is projected from a surface of the blade, an insulating derivative, for example, a fluorine resin, may be applied to the surface.

In addition, as the requirement arises, for example, a fluorine resin may be applied to the surface of the blade to ensure a dielectric withstanding voltage during sliding of the blade.

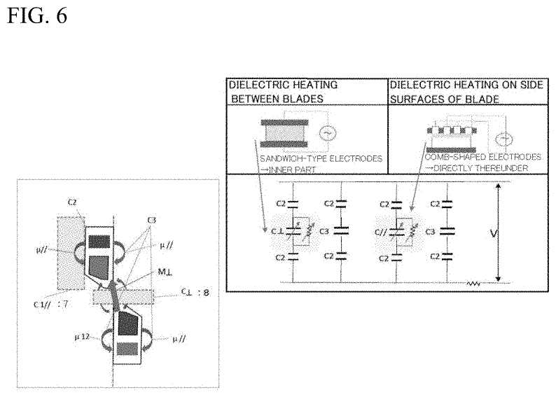

FIG. 6 can be referred to for the flow of a microwave in the first pattern of the medical treatment tool 1 according to the present invention. In this manner, a microwave flows on both side surface sides 10 of the blades and inner surface sides 11 of the blades and between the blade tips of the blade pair.

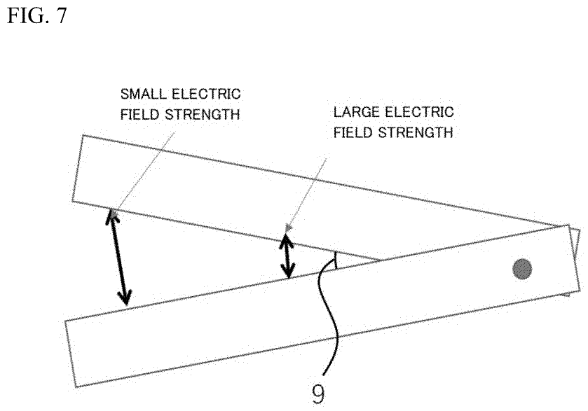

In addition, as illustrated in FIG. 7 and FIG. 8, when an opening angle 9 between the blade pair is reduced, electric field strengths at a blade base (connection portion of two electrodes or screw (fulcrum)) of the blade pair and at the blade tips of the blade pair can be maintained to be substantially constant. That is, when lengths in distal end directions (cutting edges) of the blade pair are increased to reduce an inter-blade distance of the blade pair, the electric field strengths at the blade tips and at the blade base can be brought closer to the same value. In addition, use of a blade having a small inter-blade distance enables dielectric heating (tissue fixation) to be performed swiftly.

(Medical Treatment Tool According to the Present Invention)

The medical treatment tool 1 according to the present invention has any one or more of the following effects.

The living tissue is dielectrically heated, to thereby enable fixation (protein coagulation), sealing, and hemostasis of the tissue and prevention of an outflow of body fluid from the tissue.

Through use of a high-speed dielectric heating characteristic, it is possible to incise or cut tissue including a blood vessel and a lymph vessel without bleeding and without an outflow of body fluid, for example, lymph.

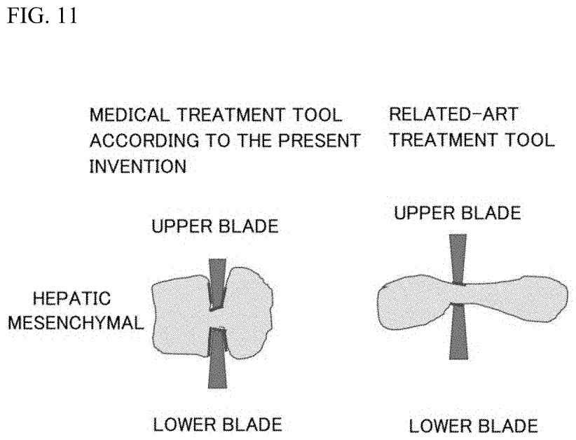

It is possible to reliably perform treatment through the fixation (protein coagulation), sealing, and hemostasis of the tissue and the prevention of body fluid outflow from the tissue. In the related-art dielectric heating of a scissors type, the tissue between the cutting edges can be uniformly heated, but the width that can be heated is narrow. The medical treatment tool 1 according to the present invention includes electrodes on the side surfaces of the blades and brings the side surfaces into abutment against the tissue, and therefore has such a feature that the tissue can be widely fixed. Specifically, as illustrated in FIG. 11, in the medical treatment tool 1 according to the present invention, unlike the related-art medical treatment tool (see the right figure of FIG. 11), the living tissue 7 can be dielectrically heated not only at the blade tips but also on the side surfaces of the blades.

The scissors type (sandwich-type electrodes) has a property of being able to perform uniform heating irrespective of a depth of the tissue, and the tweezers type (juxtaposed/parallel electrodes) has a property of being able to shallowly (relatively) heat the tissue near the electrodes. Therefore, it is possible to properly use those heating properties. In addition, the electrode can be formed to have a small diameter, and hence local side surface treatment is also possible.

It is possible to perform treatment through use of one kind of treatment tool based on the dielectric heating of the tissue nipped between the cutting edges of the blade pair and the dielectric heating of the tissue brought into abutment against electrode pairs arranged on the side surfaces of the blades, which have hitherto been performed through use of different treatment tools. The treatment through the heating between the cutting edges and the heating on the side surfaces of the blades can be performed sequentially or simultaneously, and it is not required to switch or replace the treatment tool. It is possible to obtain such an effect that the treatment can be performed appropriately and swiftly.

As described above, the medical treatment tool 1 according to the present invention can provide a treatment tool capable of not only “local tissue fixation in an incision/cutting part” but also “tissue fixation over any area” which is heating on side surfaces of electrodes of the forceps/tweezers type.

(Organic Material Coagulation Method Using Medical Treatment Tool According to the Present Invention)

In an organic material coagulation method using the medical treatment tool according to the present invention, an organic material is nipped between the blades or the jaws of the medical treatment tool according to the present invention, and then an electromagnetic wave is applied thereto, thereby being capable of subjecting the organic material to coagulation (including heating, drying, and pressurization).

(Confirmation of Effects with Prototype)

The medical treatment tool (with the coaxial cable branched off and the two branched-off destinations connected to the respective blades) capable of the microwave heating from the blade pair was compared with a medical treatment tool capable of the microwave heating from only one blade, and the following effects were confirmed.

With the medical treatment tool capable of the microwave heating from only one blade, when coagulation was performed in albumen, it was possible to confirm the coagulation only around root portions of the blade pair or the coagulation on only one of the blades that was capable of the microwave heating.

With the medical treatment tool capable of the microwave heating from the blade pair, it was confirmed that the coagulation was able to be performed on the entire surfaces along both the blades.

In clinical practice, it is known that hemostatic abilities of both an ultrasonic wave and a high-frequency device are extremely lowered in a pool of blood. However, with the medical treatment tool capable of the microwave heating from the blade pair, the coagulation can be performed in a pool of blood or in a liquid, and it is possible to suppress a decrease in hemostatic ability.

(Other Aspects of Medical Treatment Tool According to the Present Invention)

The medical treatment tool 1 according to the present invention may have the following aspects.

1. An electromagnetic wave electrode tool, including a plurality of extension members, wherein the plurality of extension members each include, on a side surface thereof along an extending direction of the extension member, a first electromagnetic wave application electrode and a second electromagnetic wave receiver electrode arranged adjacent to the first electromagnetic wave application electrode.

2. The electromagnetic wave electrode tool according to the above-mentioned item 1, wherein the plurality of extension members each include an electromagnetic wave application first electrode layer that forms the electromagnetic wave application first electrode provided in the extending direction and an electromagnetic wave receiver second electrode layer that forms the electromagnetic wave receiver second electrode provided in the extending direction.

3. A microwave electrode tool,

wherein the electromagnetic wave electrode tool according to the above-mentioned item 1 is directly or indirectly connected to a microwave coaxial cable including a central conductor and an external conductor,

wherein the first electromagnetic wave application electrode is a first microwave application electrode,

wherein the second electromagnetic wave receiver electrode is a second microwave receiver electrode, and

wherein the central conductor is directly or indirectly connected to the first microwave application electrode, and the external conductor is directly or indirectly connected to the second microwave receiver electrode.

4. A medical treatment tool, wherein the microwave electrode tool according to the above-mentioned item 3 is the medical treatment tool, the plurality of extension members includes a first blade and a second blade provided so as to be openable and closable, and the microwave application first electrode and the microwave receiver second electrode of each of the first blade and the second blade are provided at positions opposite to each other in an opening/closing direction so that a microwave can be irradiated from both the first blade and the second blade.

### INDUSTRIAL APPLICABILITY

The present invention can provide a medical treatment tool capable of heating tissue through use of a microwave from the side surfaces of the blades.

### REFERENCE SIGNS LIST

1: medical treatment tool
2: first blade (first jaw)
21: first microwave application electrode
22: first microwave receiver electrode
23: third microwave application electrode
24: third microwave receiver electrode
3: second blade (second jaw)
31: second microwave application electrode
32: second microwave receiver electrode
33: fourth microwave application electrode
34: fourth microwave receiver electrode
4: coaxial cable
41: central conductor
42: external conductor
5: insulator or dielectric body
7: living tissue subjected to microwave heating from side surfaces of blade pair
8: living tissue subjected to microwave heating from blade tips of blade pair
9: opening angle of blade pair
10: side surface side of blade pair
11: inner surface side of blade pair
12: wrist joint movement portion
13: shaft
14: power connecting portion
15: electromagnetic wave medical system
16: electromagnetic wave generating apparatus
17: drive unit
18: monitor
19: control unit

1-16. (canceled)

17. A medical treatment tool, comprising:
a first blade or a first jaw including a first electromagnetic wave application electrode and a first electromagnetic wave receiver electrode;
a second blade or a second jaw, which is arranged so as to be opposed to the first blade or the first jaw, and includes a second electromagnetic wave application electrode and a second electromagnetic wave receiver electrode; and
a coaxial cable including a central conductor and an external conductor,
wherein the central conductor is directly or indirectly connected to the first electromagnetic wave application electrode and the electromagnetic wave application electrode, and the external conductor is directly or indirectly connected to the first electromagnetic wave receiver electrode and the second electromagnetic wave receiver electrode,
wherein the first electromagnetic wave application electrode is located on an upper side of the first blade or the first jaw with respect to the first electromagnetic wave receiver electrode and the electromagnetic wave application electrode is located on an upper side of the second blade or the second jaw with respect to the second electromagnetic wave receiver electrode, or the first electromagnetic wave receiver electrode is located on the upper side of the first blade or the first jaw with respect to the first electromagnetic wave application electrode and the second electromagnetic wave receiver electrode is located on the upper side of the second blade or the second jaw with respect to the electromagnetic wave application electrode,
wherein, under a state in which the first blade or the first jaw and the second blade or the second jaw are closed,
the first electromagnetic wave application electrode and the electromagnetic wave application electrode face each other, and the first electromagnetic wave receiver electrode and the second electromagnetic wave receiver electrode face each other, and
wherein the medical treatment tool is configured to irradiate an electromagnetic wave between the first blade or the first jaw and the second blade or the second jaw, irradiate an electromagnetic wave on a side surface of the first blade or a side surface of the first jaw, and to irradiate an electromagnetic wave on a side surface of the second blade or a side surface of the second jaw.

18. A medical treatment tool, comprising:
a first blade or a first jaw including a first electromagnetic wave application electrode and a first electromagnetic wave receiver electrode;
a second blade or a second jaw, which is arranged so as to be opposed to the first blade or the first jaw, and includes a second electromagnetic wave application electrode and a second electromagnetic wave receiver electrode; and
a coaxial cable including a central conductor and an external conductor,
wherein:
the first electromagnetic wave application electrode is located on an upper side of the first blade or the first jaw with respect to the first electromagnetic wave receiver electrode and the second electromagnetic wave receiver electrode is located on an upper side of the second blade or the second jaw with respect to the electromagnetic wave application electrode, and under a state in which the first blade or the first jaw and the second blade or the second jaw are closed, the first electromagnetic wave application electrode and the second electromagnetic wave receiver electrode face each other and the first electromagnetic wave receiver electrode and the electromagnetic wave application electrode face each other; or
the first electromagnetic wave receiver electrode is located on the upper side of the first blade or the first jaw with respect to the first electromagnetic wave application electrode and the electromagnetic wave application electrode is located on the upper side of the second blade or the second jaw with respect to the second electromagnetic wave receiver electrode, and under a state in which the first blade or the first jaw and the second blade or the second jaw are closed, the first electromagnetic wave receiver electrode and the electromagnetic wave application electrode face each other and the first electromagnetic wave application electrode and the second electromagnetic wave receiver electrode face each other,
wherein the central conductor is directly or indirectly connected to the first electromagnetic wave application electrode and the electromagnetic wave application electrode, and the external conductor is directly or indirectly connected to the first electromagnetic wave receiver electrode and the second electromagnetic wave receiver electrode, and
wherein the medical treatment tool is configured to irradiate an electromagnetic wave from both the first blade or the first jaw and the second blade or the second jaw.

19. A medical treatment tool, comprising:
a first blade or a first jaw including a first electromagnetic wave application electrode, a third electromagnetic wave application electrode, a first electromagnetic wave receiver electrode, and a third electromagnetic wave receiver electrode;
a second blade or a second jaw, which is arranged so as to be opposed to the first blade or the first jaw, and includes a second electromagnetic wave application electrode, a fourth electromagnetic wave application electrode, a second electromagnetic wave receiver electrode, and a fourth electromagnetic wave receiver electrode; and
a coaxial cable including a central conductor and an external conductor,
wherein the first electromagnetic wave application electrode, the first electromagnetic wave receiver electrode, the third electromagnetic wave application electrode, and the third electromagnetic wave receiver electrode are located in the stated order from an upper side of the first blade or the first jaw and the electromagnetic wave application electrode, the second electromagnetic wave receiver electrode, the fourth electromagnetic wave application electrode, and the fourth electromagnetic wave receiver electrode are located in the stated order from an upper side of the second blade or the second jaw, and under a state in which the first blade or the first jaw and the second blade or the second jaw are closed, the first electromagnetic wave application electrode and second electromagnetic wave application electrode, the first electromagnetic wave receiver electrode and second electromagnetic wave receiver electrode, the third electromagnetic wave application electrode and fourth electromagnetic wave application electrode, and the third electromagnetic wave receiver electrode and fourth electromagnetic wave receiver electrode face each other, respectively,
wherein the central conductor is directly or indirectly connected to the first electromagnetic wave application electrode, the electromagnetic wave application electrode, the third electromagnetic wave application electrode, and the fourth electromagnetic wave application electrode, and the external conductor is directly or indirectly connected to the first electromagnetic wave receiver electrode, the second electromagnetic wave receiver electrode, the third electromagnetic wave receiver electrode, and the fourth electromagnetic wave receiver electrode, and
wherein the medical treatment tool is configured to irradiate an electromagnetic wave between the first blade or the first jaw and the second blade or the second jaw, irradiate an electromagnetic wave on a side surface of the first blade or a side surface of the first jaw, and to irradiate an electromagnetic wave on a side surface of the second blade or a side surface of the second jaw.

20. The medical treatment tool according to claim 17, wherein a part or all of the first electromagnetic wave application electrode and the first electromagnetic wave receiver electrode are built or buried in the first blade or the first jaw, and a part or all of the electromagnetic wave application electrode and the second electromagnetic wave receiver electrode are built or buried in the second blade or the second jaw.

21. The medical treatment tool according to claim 18, wherein a part or all of the first electromagnetic wave application electrode and the first electromagnetic wave receiver electrode are built or buried in the first blade or the first jaw, and a part or all of the electromagnetic wave application electrode and the second electromagnetic wave receiver electrode are built or buried in the second blade or the second jaw.

22. The medical treatment tool according to claim 19, wherein a part or all of the first electromagnetic wave application electrode, the first electromagnetic wave receiver electrode, the third electromagnetic wave application electrode, and the third electromagnetic wave receiver electrode are built or buried in the first blade or the first jaw, and a part or all of the electromagnetic wave application electrode, the second electromagnetic wave receiver electrode, the fourth electromagnetic wave application electrode and the fourth electromagnetic wave receiver electrode are built or buried in the second blade or the second jaw.

23. The medical treatment tool according to claim 17, wherein, in the first blade or the first jaw, the first electromagnetic wave application electrode and the first electromagnetic wave receiver electrode are installed through intermediation of an insulator or a dielectric body, and in the second blade or the second jaw, the electromagnetic wave application electrode and the second electromagnetic wave receiver electrode are installed through intermediation of an insulator or a dielectric body.

24. The medical treatment tool according to claim 18, wherein, in the first blade or the first jaw, the first electromagnetic wave application electrode and the first electromagnetic wave receiver electrode are installed through intermediation of an insulator or a dielectric body, and in the second blade or the second jaw, the electromagnetic wave application electrode and the second electromagnetic wave receiver electrode are installed through intermediation of an insulator or a dielectric body.

25. The medical treatment tool according to claim 19, wherein, in the first blade or the first jaw, the first electromagnetic wave application electrode, the first electromagnetic wave receiver electrode, the third electromagnetic wave application electrode, and the third electromagnetic wave receiver electrode are installed through intermediation of an insulator or a dielectric body, and in the second blade or the second jaw, the electromagnetic wave application electrode, the second electromagnetic wave receiver electrode, the fourth electromagnetic wave application electrode, and the fourth electromagnetic wave receiver electrode are installed through intermediation of an insulator or a dielectric body.

26. The medical treatment tool according to claim 17, further comprising a switch,
wherein the central conductor is directly or indirectly connected to the first electromagnetic wave application electrode, the electromagnetic wave application electrode, the third electromagnetic wave application electrode, the fourth electromagnetic wave application electrode, the fifth electromagnetic wave application electrode, and the sixth electromagnetic wave application electrode through intermediation of the switch, and the irradiation of the electromagnetic wave includes any one or more of the following items:
(1) the electromagnetic wave is irradiated between the first blade or the first jaw and the second blade or the second jaw;
(2) the electromagnetic wave is irradiated on the side surface of the first blade or the side surface of the first jaw; and
(3) the electromagnetic wave is irradiated on the side surface of the second blade or the side surface of the second jaw.

27. The medical treatment tool according to claim 17, wherein the insulator or the dielectric body comprises a fluorine resin or quartz.

28. The medical treatment tool according to claim 17, wherein the electromagnetic wave comprises a microwave.

29. The medical treatment tool according to claim 17, further comprising a grip or a trigger directly or indirectly connected to the first blade or the first jaw and/or the second blade or the second jaw,
wherein opening/closing of the first blade or the first jaw and/or the second blade or the second jaw is performed through a movement of the trigger or the grip.

30. The medical treatment tool according to claim 17, further comprising:
a wrist joint movement portion connected to a base end side of the first blade or the first jaw and the second blade or the second jaw;
a shaft connected to a base end side of the wrist joint movement portion; and
a power connecting portion connected to a base end side of the shaft,
wherein the medical treatment tool is configured to automatically perform, through power of the power connecting portion, an action of the wrist joint movement portion, an opening and closing operation of the blades or the jaws, a rotation of the shaft, and a back-and-forth movement of the shaft in a longitudinal direction.

31. An electromagnetic wave medical system, comprising:
the medical treatment tool of claim 17;
an electromagnetic wave generating apparatus configured to supply an electromagnetic wave to the treatment tool;
a drive unit configured to operate the treatment tool;
a monitor configured to present an image of the treatment tool; and
a control unit configured to control the monitor, the medical treatment tool, the electromagnetic wave generating apparatus, and the drive unit.

32. An organic material coagulation method, comprising coagulating an organic material through use of the medical treatment tool of claim 17.
